(19) United States
(12) Patent Application Publication
(10) Pub. No.: US 2021/0386736 A1
(43) Pub. Date: Dec. 16, 2021

(54) COMBINATION THERAPY FOR TREATING CANCER

(71) Applicant: AstraZeneca AB, Södertälje (SE)

(72) Inventor: Wolfram BRUGGER, Cambridge (GB)

(21) Appl. No.: 17/263,913

(22) PCT Filed: Jul. 26, 2019
(86) PCT No.: PCT/IB2019/056400

§ 371 Date: Jan. 28, 2021

Related U.S. Application Data
(60) Provisional application No. 62/711,751, filed on Jul. 30, 2018.

(51) Int. Cl. A61K 31/502; A61K 31/496; A61K 9/51; A61P 35/02

(52) U.S. Cl. CPC A61K 31/502; A61K 31/496; B82Y 5/00; A61P 35/02; A61K 9/51

(57) ABSTRACT

Disclosed are methods of treating cancer comprising administering to a subject in need thereof comprising administering to the subject an effective amount of a pharmaceutical composition comprising a plurality of AZD2811 nanoparticles and venetoclax.

### RELATED APPLICATIONS

This application claims the benefit of priority under 35 U.S.C. § 119(e) to U.S. Provisional Patent Application No. 62/711,751, filed Jul. 30, 2018, the contents of which are hereby incorporated by reference in their entirety.

### BACKGROUND

While much progress has been made in the treatment of hematological malignancies, the many of these patients who have such cancers live with an incurable disease. Those patients suffering from acute myeloid leukemia (AML) have limited treatment options, and the five-year survival rate is approximately 25% with patients over 60 responding poorly to treatment, with a median survival of less than 12 months. Accordingly, it's important to continue to find new treatments for patients with incurable cancer.

### SUMMARY

In some embodiments, disclosed is a method of treating cancer comprising administering to a subject in need thereof an effective amount of a pharmaceutical composition comprising a plurality of AZD2811 nanoparticles and an effective amount of venetoclax.

In some embodiments, disclosed is a pharmaceutical composition comprising a plurality of AZD2811 nanoparticles for use in the treatment of cancer, wherein said treatment comprises the separate, sequential or simultaneous administration of venetoclax.

In some embodiments, disclosed is venetoclax for use in the treatment of cancer, wherein said treatment comprises the separate, sequential or simultaneous administration of a pharmaceutical composition comprising a plurality of AZD2811 nanoparticles.

In some embodiments, disclosed is a kit comprising: a first pharmaceutical composition comprising a plurality of AZD2811 nanoparticles and a pharmaceutically acceptable carrier; and a second pharmaceutical composition comprising venetoclax, and instructions for use.

### BRIEF DESCRIPTIONS OF THE DRAWINGS

FIG. 1 illustrates the KG1a tumor volume over time of mice treated with vehicle, AZD2811 alone, venetoclax alone (ABT-199), and a combination of AZD2811 and venetoclax (ABT-199).

FIG. 2 illustrates the HL-60 tumor volume over time of mice treated with vehicle, different doses of AZD2811 alone, venetoclax alone (ABT-199), and a combination of different doses of AZD2811 and venetoclax (ABT-199).

FIG. 3 illustrates the statistically significant (p=0.01; Log Rank test) survival benefit of combining AZD2811 with venetoclax (ABT-199) over the standard-of-care combination of venetoclax (ABT-199) and 5-azacitidine in the disseminated AML xenograft model MOLM-13

### DETAILED DESCRIPTION

In some embodiments, disclosed is a method of treating cancer comprising administering to a subject in need thereof an effective amount of a pharmaceutical composition comprising a plurality of AZD2811 nanoparticles and an effective amount venetoclax.

The language “AZD2811 nanoparticles” includes nanoparticles that comprise the Aurora kinase B inhibitor 2-(3-((7-(3-(ethyl(2-hydroxyethyl)amino)propoxy)quinazolin-4-yl)amino)-1H-pyrazol-5-yl)-N-(3-fluorophenyl)acetamide (also known as AZD1152 hqpa), about 7 to about 15 weight percent of pamoic acid, and a diblock poly(lactic) acid-poly(ethylene)glycol copolymer; wherein the diblock poly(lactic) acid-poly(ethylene)glycol copolymer has a poly(lactic acid) block having a number average molecular weight of about 16 kDa and a poly(ethylene)glycol block having a number average molecular weight of about 5 kDa; wherein the poly(ethylene)glycol block comprises about 10 to 30 weight percent of the therapeutic nanoparticle. Preparation of the AZD2811 nanoparticles is disclosed in International Application Publication No. WO2015/036792.

Venetoclax (also known as ABT-199) is a BCL-2 inhibitor approved for the treatment of patients with chronic lymphocytic leukemia (CLL) or small lymphocytic lymphoma (SLL), with or without 17p deletion, who have received at least one prior therapy. Venetoclax has the structure below and is disclosed as Example 5 in International Application Publication No. 2010/138588:

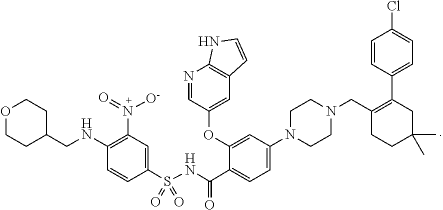

In some embodiments, venetoclax is orally administered. In some embodiments, venetoclax is administered as an oral pharmaceutical composition comprising 10 mg, 50 mg or 100 mg of venetoclax. In some embodiments, venetoclax is administered at a 20 mg dose once daily for 7 days, followed by a weekly ramp-up dosing schedule over four weeks to a daily dose of 400 mg.

The language “treat,” “treating” and “treatment” includes the reduction or inhibition of enzyme or protein activity related to Aurora kinase B, BCL-2 or cancer in a subject, amelioration of one or more symptoms of cancer in a subject, or the slowing or delaying of progression of cancer in a subject. The language “treat,” “treating” and “treatment” also includes the reduction or inhibition of the growth of a tumor or proliferation of cancerous cells in a subject.

The language “inhibit,” “inhibition” or “inhibiting” includes a decrease in the baseline activity of a biological activity or process.

The term “cancer” includes, but is not limited to, hematological malignancies, such as acute myeloid leukemia (AML), MDS, CMML, multiple myeloma, mantle cell lymphoma (MCL), chronic lymphocytic leukemia (CLL), diffuse large B cell lymphoma (DLBCL), Burkitt's lymphoma, follicular lymphoma and small lymphocytic lymphoma (SLL). In some embodiments, the cancer is a cancer susceptible to an Aurora kinase B inhibitor (e.g., AZD2811 nanoparticles). In some embodiments, the cancer is a cancer susceptible to a BCL-2 inhibitor (e.g., venetoclax).

The term “subject” includes warm-blooded mammals, for example, primates, dogs, cats, rabbits, rats, and mice. In some embodiments, the subject is a primate, for example, a human. In some embodiments, the subject is suffering from cancer. In some embodiments, the subject is in need of treatment (e.g., the subject would benefit biologically or medically from treatment).

The language “pharmaceutical composition” includes compositions comprising a plurality of AZD2811 nanoparticles and a pharmaceutically acceptable excipient, carrier or diluent. The language “pharmaceutically acceptable excipient, carrier or diluent” includes compounds, materials, compositions, and/or dosage forms which are, within the scope of sound medical judgment, suitable for use in contact with the tissues of human beings and animals without excessive toxicity, irritation, allergic response, or other problem or complication, as ascertained by one of skill in the art. Pharmaceutical compositions may be in the form of a sterile injectable solution in one or more aqueous or non-aqueous non-toxic parenterally-acceptable buffer systems, diluents, solubilizing agents, co-solvents, or carriers. A sterile injectable preparation may also be a sterile injectable aqueous or oily suspension or suspension in a non-aqueous diluent, carrier or co-solvent, which may be formulated according to known procedures using one or more of the appropriate dispersing or wetting agents and suspending agents. The pharmaceutical compositions could be a solution for iv bolus/infusion injection or a lyophilized system (either alone or with excipients) for reconstitution with a buffer system with or without other excipients. The lyophilized freeze-dried material may be prepared from non-aqueous solvents or aqueous solvents. The dosage form could also be a concentrate for further dilution for subsequent infusion.

The language “effective amount” includes that amount of a pharmaceutical composition comprising AZD2811 nanoparticles and/or that amount of venetoclax that will elicit a biological or medical response in a subject, for example, the reduction or inhibition of enzyme or protein activity related to Aurora kinase B, BCL-2 or cancer; amelioration of symptoms of cancer; or the slowing or delaying of progression of cancer. In some embodiments, the language “effective amount” includes the amount of a pharmaceutical composition comprising AZD2811 nanoparticles and/or venetoclax, that is effective to at least partially alleviate, inhibit, and/or ameliorate cancer or inhibit Aurora kinase B, BCL-2, and/or reduce or inhibit the growth of a tumor or proliferation of cancerous cells in a subject.

In some embodiments, disclosed is a kit comprising: a first pharmaceutical composition comprising a plurality of AZD2811 nanoparticles and a pharmaceutically acceptable carrier; and a second pharmaceutical composition comprising venetoclax and instructions for use.

### EXAMPLES

### Example 1: Efficacy of AZD2811, a Selective AURKB Inhibitor, Combined with Venetoclax in a Preclinical Model of Acute Myeloid Leukemia

KGa1: 2×10⁷ KG1a AML cells in 50% matrigel were implanted subcutaneously on the left flank of adult female SCID mice. Mice were randomised by tumor volume at D7 into groups of 8, with an average tumor volume of 0.2 cm³ and all dosing was started. AZD2811 nanoparticles were dosed at once weekly with a 20-30 s intravenous infusion at either 100 mg/kg (100 mg/kg was the maximum tolerated dose in combination with venetoclax (ABT-199) 100 mg/kg; venetoclax was administered orally daily at 100 mg/kg). All drugs were given for 3 weekly cycles.

HL-60: 1×10⁷ HL-60 AML cells in 50% matrigel were implanted subcutaneously on the left flank of adult female SCID mice. Mice were randomised by tumor volume at D7 into groups of 8, with an average tumor volume of 0.2 cm³ and all dosing was started. AZD2811 nanoparticles were dosed at once weekly with a 20-30 s intravenous infusion at either 50 mg/kg, 25 mg/kg, 12.5 mg/kg and 6.25 mg/kg (100 mg/kg was the maximum tolerated dose in combination with venetoclax (ABT-199) 100 mg/kg; venetoclax was administered orally daily at 100 mg/kg). All drugs were given for 3 weekly cycles. For both models, tumors were measured twice weekly by single operators, and all dosing was performed by randomised cage to minimise systematic bias. For the MOLM-13 orthotopic model, 1×10⁶ MOLM-13 cells were injected into the tail vien of adult female NOG mice. After 3 days, mice were randomised by body-weight into groups of 8 and treatment was commenced the following day. AZD2811 nanoparticles were dosed at once weekly with a 20-30 s intravenous infusion at 25 mg/kg (25 mg/kg was the maximum tolerated dose in combination with venetoclax (ABT-199) 100 mg/kg; venetoclax was administered orally daily at 100 mg/kg). 5-azacytidine was dosed twice-daily at 0.5 mg/kg for three days by the intraperitoneal route, followed by 4 rest days of no dosing; 5-azacutidine was dosed in combination with venetoclax at 100 mg/kg daily. All drugs were given for 2 weekly cycles, the study endpoint was determined by a welfare scoring table.

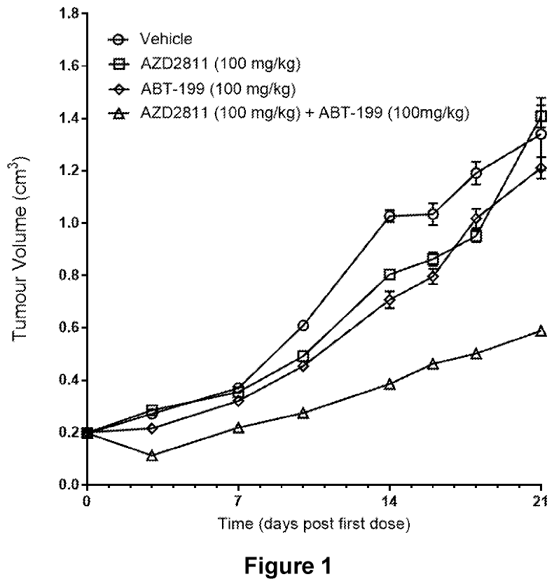

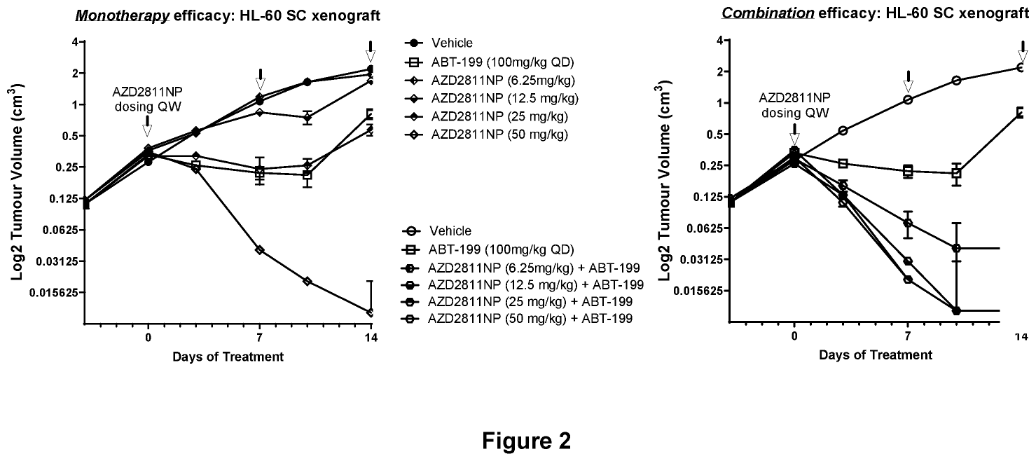

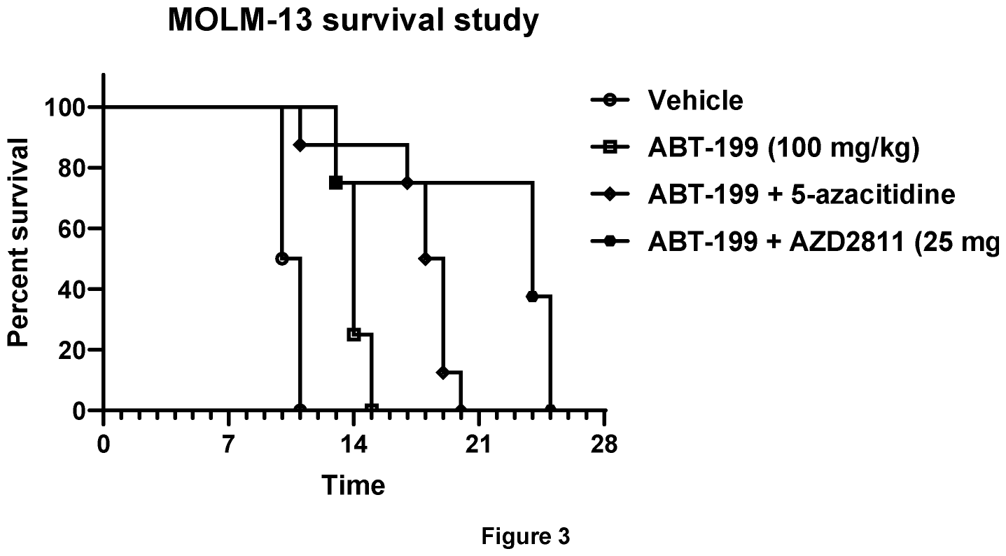

Results: As shown in FIG. 1, both AZD2811 nanoparticle and venetoclax (ABT-199) monotherapy were modestly efficacious in the KG1a model, and the combination with venetoclax showed considerably enhanced efficacy in combination as compared to either single agent alone. As shown in FIG. 2, both AZD2811 nanoparticle and venetoclax (ABT-199) monotherapy were active in the HL-60 model, and the combination with venetoclax showed considerably enhanced efficacy in combination as compared to either single agent alone, and notably at the lowest doses of AZD2811. In FIG. 3, the combination of AZD2811 and venetoclax delivered a statistically significant survival benefit over the standard-of-care regiment venetoclax and 5-azacitidine, which in itself improved survival over venetoclax alone.

1. A method of treating cancer comprising administering to a subject in need thereof an effective amount of a pharmaceutical composition comprising a plurality of AZD2811 nanoparticles and an effective amount of venetoclax.

2. The method of claim 1, wherein the method comprises administering the pharmaceutical composition comprising a plurality of AZD2811 nanoparticles sequentially, separately or simultaneously with venetoclax.

3. The method of claim 1, wherein the cancer is a hematological cancer.

4. The method of claim 3, wherein the hematological cancer is selected from acute myeloid leukemia (AML), MDS, CMML, multiple myeloma, mantle cell lymphoma (MCL), chronic lymphocytic leukemia (CLL), diffuse large B cell lymphoma (DLBCL), Burkitt's lymphoma and follicular lymphoma.

5. A pharmaceutical composition comprising a plurality of AZD2811 nanoparticles for use in the treatment of cancer, wherein said treatment comprises the separate, sequential or simultaneous administration of venetoclax.

6. Venetoclax for use in the treatment of cancer, wherein said treatment comprises the separate, sequential or simultaneous administration of a pharmaceutical composition comprising a plurality of AZD2811 nanoparticles.

7. The use of claim 4 or 5, wherein the cancer is a hematological cancer.

8. The use of claim 7, wherein the hematological cancer is selected from acute myeloid leukemia (AML), MDS, CMML, multiple myeloma, mantle cell lymphoma (MCL), chronic lymphocytic leukemia (CLL), diffuse large B cell lymphoma (DLBCL), Burkitt's lymphoma and follicular lymphoma.

9. A kit comprising:
a first pharmaceutical composition comprising a plurality of AZD2811 nanoparticles and a pharmaceutically acceptable carrier; and
a second pharmaceutical composition comprising venetoclax and a pharmaceutically acceptable carrier.
